(19) United States
(12) Patent Application Publication
(10) Pub. No.: US 2002/0072646 A1
(43) Pub. Date: Jun. 13, 2002

(54) Use of magnetic fields to enhance immune system performance

(76) Inventor: Van R. Irion, Durham, CA (US)

Correspondence Address: EMF Biotechnologies Inc., 1884 Cummings Lane, Durham, CA, 95938, US

(21) Appl. No.: 09/978,222

(22) Filed: Oct. 16, 2001

Related U.S. Application Data
(60) Provisional application No. 60/108,581, filed on Nov. 16, 1998.
(60) Provisional application No. 60/236,801, filed on Oct. 2, 2000.

(51) Int. Cl. A61N 2/00

(52) U.S. Cl. 600/9; 607/2

(57) ABSTRACT

Low energy alternating current magnetic fields are used to induce genes that regulate the cellular stress response and the immune response. This method of gene induction causes little or no damage to the cell. The cascade of repair and immune genes induced helps prevent tumor genesis and slow tumor growth. These genes can be repeatedly induced, in the subjects' entire body, for the purpose of increasing immune response and/or increasing survival from infectious disease.

### CROSS REFERENCE TO RELATED APPLICATIONS

This non-provisional application contains information contained in provisional application Ser. No. 60/108,581, filed on Nov. 16, 1998, provisional application Ser. No. 60/236,801, filed on Oct. 2, 2000, and non-provisional application Ser. No. 09/431,408, filed of Oct. 30, 1999.

No other related applications.

### STATEMENT REGARDING FEDERALLY SPONSORED RESEARCH OR DEVLOPMENT

Not Applicable.

### REFERENCE TO A MICROFICHE APPENDIX

Not Applicable.

### BACKGROUND OF THE INVENTION

First, I will give a brief foundational review of information and scientific studies which lead to this application. The methods contained in this application span the fields of molecular/cellular biology, medicine, and magnetic field physics.

Recent studies have suggested that continuous or repeated induction of the cellular stress response could lead to increased longevity and increased resistance to disease. The cellular stress response evolved as a method of repairing damage to cells after a near lethal insult. After severe damage to the cell, genes encoding a cascade of repair proteins are quickly “turned on”. These repair proteins help the cell recover from the damage incurred. Many genes involved in the stress response cascade are also involved in regulating the immune response. When the stress response is induced, the immune system is also up-regulated.

Unfortunately until now, all known methods of inducing the stress response cause damage to the cell. This damage, for most methods, is too great to warrant its use for treating most pathologies. Until now...

A new method for inducing the stress response has been found. Goodman and Blank report induction of the stress response via exposing tissue to low energy magnetic fields. The methods described involve exposing tissue to specific alternating current electromagnetic fields. They used various magnetic field parameters under 1 Gauss and less than 300 Hertz. This method causes little or no damage to the cell on the molecular level. In comparison to thermal stress (the classic stress response induction method), magnetic fields induce similar stress responses at energies which are fourteen orders of magnitude lower. For more information see reference number 77.

Goodman and Blank have never published any statement or made any claim suggesting the use of magnetic fields to induce the stress response for the purpose of increasing immune response. Nor have any others to my knowledge. Publications of Goodman and Blank's research have been specifically focused on using this new method of stress response induction to pre-protect tissues and organs prior to a scheduled surgery. Induction of the stress response prior to an injury has been shown to increase the cells' ability to survive the subsequent injury.

This patent application is unique in that it couples the use of alternating current magnetic fields for repeatedly inducing the immune system. This is a unique application of the techniques described by Goodman and Blank. The use of the stress response to pre-protect tissue from subsequent damage involves a one-time treatment of specific tissue, and is indicated for protection against gross injury. My application calls for the exposure of all bodily tissue to low energy magnetic fields on a repeated basis, possibly for the lifetime of the subject. My methods are intended to increase the patient's resistance to disease and increase overall immune response.

The following paragraphs describe support for an earlier patent of mine that proposes chronic induction of the cellular stress response to slow the aging process. I include this because these studies also support the theory that magnetic fields can increase immune response, and help immune compromised patients. As stated earlier, the stress response cascade includes the induction of genes that regulate immune response. Also, the repair proteins induced by the stress response can help increase overall survival of the patient by increasing the survival individual cells under stress. This already been tested in whole animals. Pre-treatment with magnetic fields has already exhibited the ability to increase survival of experimental animals under near lethal stress by at least 73%. (Specifics of my studies to date are described at the end of this section.) These results are likely to translate into increased survival in cells under stresses such as bacterial infection.

The evidence supporting the efficacy of this application is extensive. Dozens of independent studies dating back to 1917 have reported increased longevity in various species, under various experimental conditions. All of these studies had one thing in common: Chronic, systemic stress in the longer living animals. Again, it is my hypothesis that the cellular repair mechanism referred to as the stress response also repairs some amount of incidental environmental damage associated with aging. Others have since proposed this hypothesis. None have suggested coupling it with magnetic induction of the stress response.

Many models of aging have developed in the past several decades. Mitocondrial defects, calcium mis-metabolism, collagen loss, free-radical damage, suicide genes, and somatic cell mutation, have all been proposed as primary causes of aging. I propose that all of these factors play a roll in the slow but cumulative degeneration of the body. The chronic terminal syndrome we collectively call “aging” is a result of constant environmental insults on a molecular level. Solar radiation, oxidation, and accumulation of toxins are the primary causes. The damage incurred by these insults is additive. Over a lifetime they slowly overwhelm the body's ability to repair it self. Toward the end of an animal's natural life span, the damage is often so great that its immune system's ability to self-recognize begins to fail. When this occurs, a myriad of age related autoimmune syndromes are likely to arise, and incidences of cancer increase.

The best defense against this syndrome is continual damage control on a molecular level. Many types of constitutive cellular maintenance have evolved. Unfortunately, these mechanisms only improve short-term survival. Evolution only improves survival up to and through the age of optimal reproduction. Any mutation, which improves odds of survival after reproduction, has as good a chance of being eliminated through evolution as it has of being conserved. This has resulted in cellular maintenance mechanisms, which allow rapid accumulation of incidental damage over time. This accumulation of damage further decreases the ability of the body to repair itself, and even more damage is accumulated. Existing, constitutive cellular repair mechanisms alone are not enough to prevent aging. Acute repair mechanisms, which repair cellular damage at a much faster rate, would be required to slow or reverse the aging syndrome.

These acute cellular repair mechanisms do exist. They are the cascade of repair proteins created as a result of the cellular stress response mentioned earlier. Ritossa first documented evidence of an acute stress response on a genetic level in 1962. Over the next three and a half decades the stress response and the cascade of repair proteins, which it evokes, have been continuously studied. Two primary end products of this cascade are Heat Shock Protein 72 (HSP72) and Super Oxide Dismutase (SOD). These acute repair proteins are expressed under conditions of extreme stress to the cell. Anoxia or hyperoxia, hyper or hypothermia, toxins, and starvation are known to evoke the stress response.

Unfortunately, the near lethal conditions required to evoke the stress response most likely cause enough molecular damage to the cell that any net gains, due to the production of repair proteins, would be minimal. Prior to the studies reported in this application, the only three methods found that consistently extends the maximum life span involve extreme caloric restriction, introduction of toxic substances, or constant thermal stress. While caloric restriction and toxic substances have proven to have serious side effects, thermal stress is better tolerated. This method is likely to cause systemic stress that is severe enough to provoke a chronic cellular stress response, while causing minor molecular damage. This would allow for a net gain in cellular damage control over normal environmental conditions.

In 1917 researchers at the Rockefeller Institute extended the life span of fruit flies by keeping them at 19° C. rather than the normal 25° C. In 1943 McCay et al were able to more than double the maximum life span of rats via calorie restricted diet. In 1972 Liu et al reported a 75% increase in the life span of fish kept at 15° C. over fish kept at 20° C. Ethoxyquin increased longevity in mice in 1971; Thiazolidine increased lifespan of mice in 1979; Sulfhydryl containing compounds increase longevity in mice, rats and Guinea pigs, 1965; butylated hydroxytoulene (BHT) increased longevity in mice, 1979. These are just a few of the dozens of studies reporting increased longevity. All of these methods either evoke a systemic stress response or directly deliver antioxidants, or both. Yet none produce more than a minor increase in longevity because all these methods create almost as much damage as they repair through the stress response . . . minimal net gain.

Studies investigating the molecular effects of stress most often focus on molecular chaperones like HSP72, or free radical scavengers like SOD, all of which are highly conserved through evolution. Though the stress response induces production of many different proteins, HSP72 and SOD are two of the primary end products of this cascade. HSP72 is likely to have many functions in cellular damage repair. It is known to prevent aggregation/precipitation of unfolded proteins and aid in their transport and re-folding. HSP72 is also involved in the import of nuclear proteins to the inner mitochondrial matrix. These tasks not only replenish metabolic function quickly, they save relatively large amounts of energy which would be required to re-synthesize the denatured protein. SOD and other free-radical scavengers minimize the damage of stress conditions by reducing free-radical species, which are produced in excess during stress.

A direct link between stress genes and longevity was reported by Lithgow et al in 1996. In this report Lithgow describes several Caenorhabditis elegans mutants that exhibit extended life span and stress resistant phenotypes. Two earlier studies by Lithgow et al report increased longevity of non-mutant Caenorhabditis elegans under conditions of thermal stress.

Numerous studies have shown that the ability to produce stress proteins decreases dramatically with age. Larsen reported diminishing levels of Super Oxide Dismutase with age, in wild type Caenorhabditis elegans compared to a long-lived mutant. Heydari et al not only found a diminished ability to produce Heat Shock Protein 72 in older rats, they also found that caloric restriction over a lifetime reversed this effect. These studies support our proposed explanation of increased longevity in calorie restricted rats and also indicates that a chronic stress response and increased immune response can be maintained for extended periods of time.

Summary of my Longevity Studies to Date:

Once again, the methods described in this patent application have been reduced to practice. I have performed several studies on the nematode species Caenorhabditis elegans. This species of small roundworm are native to soil in temperate latitudes all around the world. It is a short-lived animal that has been used in longevity studies for many years.

The studies described in this section utilized two, ten-wrap, 30 cm diameter helmholtz coils, attached to an alternating current transformer. The helmholz coils were aligned on a cylinder with 30 cm separation. The coils were connected to an alternating current transformer, which allowed for the adjustment of current to create an 80 miliGauss field. This field strength was verified using a hand held Gauss meter. The current used to achieve this field strength agreed with previously calculated quantities. Alternating current cycles used were 60 Hertz.

Experimental groups of C. elegans were exposed to 80 mG/60 Hz AC fields for 20 minutes every 24 hours of their lives. Negative control groups were maintained under identical conditions to experimental groups with the exception of lack of daily exposure to the magnetic fields. Upon death, individual worms were removed and date and time were recorded. After all worms had died, average life spans were calculated by adding the total number of days lived by the group and dividing by the number of individuals in the group at the beginning of the experiment.

This experiment has been repeated several times with slight variations in maintenance temperature and group sizes. At an average maintenance temperature of 15° C. the experimental groups to date have had an average life span 10.0% longer than the negative control groups. At an average maintenance temperature of 25° C. the experimental groups to date have had an average life span 16.7% longer than the negative control groups.

Summary of survival studies:

The fruit fly Drosophila Melanogaster was used to test for increased survival of near lethal heat stress after exposed to magnetic fields. The experimental groups (totaling 88 individual flies in three separate experiments) were exposed to 80 mG/60 Hz AC fields for 20 minutes, six hours prior to near lethal heat stress. Positive control groups (totaling 94 individual flies in three separate experiments) were exposed to 38° C. temperatures for 20 minutes, six hours prior to near lethal heat stress. Negative control groups (totaling 105 individual flies in three separate experiments) were maintained under identical conditions as the other groups with the exception of the absence of magnetic fields or heat pre-treatment. At six hours post-exposure, all three groups were exposed to 41° C. for 20 minutes. Survival of magnetic field exposed flies averaged 86%. Survival of heat pre-treated flies averaged 78%. Survival of control flies averaged only 13%. These startling results emphasize the profound biological effect of electromagnetic fields.

The magnetic field parameters used in these studies have not been optimized. Larger differences in longevity are expected with further studies. Parameters such as magnetic field strength, cycle speed, exposure time, and exposure frequency are currently being tested. These will quickly be followed by longevity studies in mice.

More specific data, graphs, and/or copies of notebooks can be forwarded if requested.

Inducing a cascade of repair genes via magnetic field exposure represents a npn-invasive method of slowing the degenerative effects of aging. This method of continuous cellular damage control is analogous to Dr. Jonas Salk's use of inoculation to induce the immune system. Both use an existing cellular defense system that evolved over millions of years. Both simply turn on those systems on at a more useful time. The medical benefits resulting from methods described in this application will be as far reaching as those following Dr. Salk's discovery of immunization.

References:

1. Saul Kent, The Life Extension Revolution, 1978 p.60-1

2. Novelli, et al, Oxygen free radicals in shock, International Workshop, Florence, 1985 p.119-24 (Karger, Basel 1986)

3. Comfort A. et al, Effect of Ethoxyquin on the longevity of C3H mice, Nature, (229) 5282

4. Oeriu S. et al, The effect of the administration of compounds which contain sulfhydryl groups on the survival rates of mice, rats, and guinea pigs, J. Gerontology (20)3

5. Neal K. et al, Effects of the antioxidant butalated hydroxytoluene (BHT) on mortality in BALBIc mice, J. Gerontology (34)4

6. Kong D. et al, Expression of heat shock protein 70 but not c-fos in an anoxia tolerant turtles' brain during prolonged hypoxia, Anesthesia & Analgesia (84)S363

7. Langer T. et al, Chaperone function on crete: a meeting report, Cell Stress & Chaperones 1 (1)5-12

8. Schochetman G. et al, Characterization of the messenger RNA release from L cell polyribosomes as a result of temperature shock, J. Mol. Biol (63)577-90

9. Raven P. et al, Thermal, metabolic, and cardiovascular responses to various degrees of cold stress, Can. J. Physiol. Pharmacol (53)293-8

10. Tavaria M. et al, A hitchhiker's guide to the human HSP70 family, Cell Stress & Chaperones 1(1)23-8

11. Wynn R. et al, Molecular chaperones: heat shock proteins, foldases, and matchmakers, J. Lab Clin. Med. (124)1 31-6

12. Hendrick J. et al, The role of molecular chaperones in protein folding, FASEB Journal (9)1559-68

13. Ruis H. et al, Stress signaling in yeast, BioEssays (17) 11 959-65

14. Kenward N. et al, Heat shock proteins, molecular chaperones and the prion encephalopathies, Cell Stress & Chaperones 1(1)18-22

15. Cullen K. et al, Characterization of hypothermia-induced cellular stress response in mouse tissues, J. Bio. Chem. (272)3 1742-6

16. Nwaka S. et al, The heat shock factor and mitochondrial HSP 70 are necessary for survival of heat shock in Saccharomyces cerevisiae, FEBS Letters (399)259-63

17. Liu X. et al, Oxidative stress induces heat shock factor phosphorylation and HSF-dependant activation of yeast metallothionein gene transcription, Genes & Development (10)592-603

18. Aldridge S. Hungary-based company biorex builds its future on heat shock proteins, Genetic Engineering News 9/1/97 24

19. Liu R. et al, Potential involvement of a constitutive heat shock element binding factor in the regulation of chemical stress induced hsp70 gene expression, Mol. Cell Biochemistry (144)27-34

20. Okinaga S. et al, Identification of a nuclear protein that constitutively recognizes the sequence containing a heat shock element, Eur. J. Biochem (212)167-75

21. Orosz A. et al, Regulation of drosophila heat shock factor trimerization: global sequence requirements and independence of nuclear localization, Mol. Cell Biol. (16)12 7018-30

22. Lee Y. et al, Homoharringtonine induces heat protection and facilitates dissociation of heat shock transcription factor and heat shock element complex, Biochemical Res. Comm. (197) 2 1011-8

23. Voellmy R., Review of patents in the cell stress and chaperone field, Cell Stress & Chaperones 1(1)29-32

24. Feder J. et al, The consequences of expressing hsp 70 in drosophila cells at normal temperatures, Genes & Development 1992 p.1402-13

25. Lithgow G. et al, Thermotolerance and extended life-span conferred by single gene mutations and induced by thermal stress, PNAS (92) 7540-4

26. Lithgow G. et al, Thermotolerance of a long-lived mutant of caenorhabditis elegans, J. Gerontology (49) 6 B270-6

27. Lithgow G. et al, Mechanisms and Evolution of Aging, Science (273)80

28. Cortopassi G. et al, There is substantial agreement among interspecies estimates of DNA repair activity, Mech Ageing Dev. (91)3 211-8

29. Holmquist G. et al, Somatic mutation theory, DNA repair rates, and the molecular epidemiology of p53 mutations, Mutation Res. (386)1 69-101

30. Ashcroft G. et al, Aging is associated with reduced deposition of specific extracellular matrix components, an upregulation of angiogenesis, and an altered inflammatory response in a murine incisional wound healing model, J. Invest. Dermatol. (108)4 430-7

31. Ying W., Deleterious network hypothesis of aging, Med. Hypotheses (48)2 143-8

32. de Gray A., A proposed refinement of the mitochondrial free readical theory of aging, Bioessays (19)2 161-6

33. Hiley P., The thermoregulatory responses of the galago, the baboon, and the chimpanzee to heat stress, J. Physiol. (254)657-71

34. Narendranath R. et al, Effects of heat stress in rats, Ind. J. Physiol. Pharmac. (19)3 140-5

35. Kanwar K. et al, Chromatographic analysis of protein hydrolysates of tar testis after heat shocks, Fertility and Sterility (23)3 182-5

36. Wildt D, et al, Physiological temperature response and embryonic mortallity in stressed swine, Amer. J. Physiol. (229)6 1471-5

37. McKenzie S. et al, Translation in vitro of drosophila heat shock messages, J. Mol. Biol. (117)279-83

38. Rittossa F., Behaviour of RNA and DNA synthesis at the puff level in salivary gland chromosomes of drosophila, Exper. Cell Res. (36)515-23

39. Mirault M. et al, The effect of heat shock on gene expression in drosophila melanogaster, 819-27

40. Bouch G. et al, Effects of heat shock on gene expression and subcellular protein distribution in chinese hamster ovary cells, Nucleic Acida Res. (7)7 1739-47

41. Murphy P. et al, Nonsteroidal anti inflammatory drugs alter body temperature and supress melatonin in humans, Physio. & Behavior (59)1 133-9

42. Myers R. et al, Role of brain Ca+2 in central control of body temperature during exercise in the monkey, J. Applied Physiol. (43)4 689-94

43. Ahmad R. et al, Expression of heat shock protein 70 is altered by age and diet at the level of transcription, Mol. Cell Biol. (13)5 2909-18

44. Ceballos-Picot I. Et al, Age related changes in antioxidant enzymes and lipid peroxidation in breins of control and transgenic mice overexpressing copper-zinc superoxide dismutase, Mutation Res. (275)281-93

45. Orr W. et aL Extension of Life span by overexpression of superoxide dismutase and catalase in drosophila melanogaster, Science (263)1128-30

46. Orr W. et al, Effects of Cu-Zn superoxide dismutase overexpression on life span and resistance to oxidative stress in transgenic drosophila melanogaster, Archives of Biochem & Biophys (301)1 34-40

47. Shikama N. et al, Protein synthesis elongation factor EF-1 expression and longevity in drosophila melanogaster, PNAS (91)4199-4203

48. Larsen P., Aging and resistance to oxidative damage in caenorhabditis elegans, PNAS (90)8905-9

49. Pahlavani M et al, The expression of heat shock protein 70 decreases with age in lymphocytes from rats and rhesus monkeys, Experim. Cell Res. (218)310-8

50. Fleming F. et al, Role of oxidative stress in drosophila aging, Mutation Res. (275)276-9

51. Spradling A. et al, Messenger RNA in heat shocked drosophila cells, J. Mol. Biol. (109)559-87

51. Tissieres A. Protein synthesis in salivary glands of drosophila melanogaster: relation to chromosome puffs, J. Mol. Biol. (84)389-98

52. Plumier J. et al, Transgenic mice expressing the human heat shock protein 70 have improved post isvhemic myocardial recovery, J. Clin. Invest. (95)1854-60

53. Myers R. and Yaksh T., Thermoregulation around a new set point established in the monkey by altering the ratio of sodium to calcium ions within the hypothalamus, J. Physiol. (218)609-33

54. Myers R. et al, Species continuity in the thermoregulatory responses of the pigtailed macaque to monoamines injected into the hypothalamus, Comp. Biochem. Physiol. (51A)639-45

55. Plumier J. et al, Heat shock induced myocardial protection against ischemic injury: a role for HSP70?, Cell Stress & Chaperones 1(1)13-17

56. Maulik N. et al, Improved postischemic ventricular functional recovery by amphetamine is linked with its ability to induce heat shock, Mol. & Cell Biochem (137)17-24

57. Amrani M. et al, Kinetics of induction and protective effect of heat shock proteins after cardioplegic arrest, Ann. Thorac. Surg. (61)1407-12

58. Soncin F. et al, Reciprocal effects of pro inflammatory stimuli and anti inflammatory drugs on the activity of heat shock factor 1 in human monocytes, Biochem. & Biophys. Res. Comm.(229)479-84

59. Kamano H. et al, B-Myb and cyclin D1 mediate heat shock element dependent activation of the human hsp70 promotor, Oncogene (14)1223-29

60. Nakai, A, et al, HSF4, a new member of the human heat shock factor family which lacks properties of a transcriptional activator, Mol. Cell Biol. (17)1 469-81

61. Liu R. et al, Dual control of heat shock response: involvement of a constitutive heat shock element binding factor, PNAS (90)3078-82

62. Erkine A. et al, Heat shock factor gains access to the yeast HSC82 promoter independently of other sequence specific factors and antagonizes nuleosomal repression of basal and induced transcription, Mol. Cell Biol. (16)12 7004-17

63. Dale, E. et al, Claning and characterization of the promoter for murine 84-kDa heat shock protein, Gene (172)279-84

64. Pahlavani M. et al, The expression of heat shock protein 70 decreases with age in lymphocytes from rats and rhesus monkeys, Experi. Cell Res. (218)310-8

65. Chrisp C. et al, Lifespan and lesions in genetically heterogeneous mice: a new model for aging research, Vet. Pathol. (33)735-43

66. Mestril R. et al, Adenovirus mediated gene transfer of a heat shock protein 70 protects against simulated ischemia, J. MoL Cell Cardiol. (28)2351-8

67. Yang et al, Heat shock protein induction extends survival of rats with heatstroke, Society for Neuroscience (23) 848.14

68. Rajdev et al, Heat shock protein 70 expression in brains of transgenic mice overexpressing the rat inducible HSP70 protection against ischemia, Society for Neuroscience (23) 848.15

69. Harrub et al, Cryptic expression of the HSP72 after transient ischemia, Society for Neuroscience (23) 848.17

70. Chou et al, HSP70 prevents corticosterone from aggravating ischemic cell death, Society for Neuroscience (23) 848.18

71. Xu et al, HSP70 overexpression in astrocytes protects neurons from glucose deprivation, Society for Neuroscience (23) 845.13

72. Beaucamp et al, Effect of human inducible HSP70 on refolding of firefly luciferase in vitro and in vivo, Society for Neuroscience (23) 729.16

73. Yenari, Sharp et al, Overexpression of HSP70 using herpes simplex viral vectors improves neuron survival in experimental stroke, Society for Neuroscience (23) 547.12

74. Rankin et al, Heat shock and cold shock differentially affect long term memory consolidation of habituation in caenorhabditis elgans, Society for Neuroscience (23) 525.15

75. Guhl et al, HSP72 expression in gastric mucosa of an animal model of anorexia nervosa, Society for Neuroscience (23) 102.17

76. Johnson, Lithhow, Murakami, Hypothesis: Interventions that Increase the Resopnse to Stress Offer the potential for Effectiove Life Prolongation and Increased Health, Journal of Gerontology: Biological Sciences, '96, Vol. 51A, No. 6, p.B392-B395

77. Goodman and Blank, Magnetic Field Stress Induces Expression of Hsp70, Cell Stress & Chaperones, '98, 3(2), p.79-88.

### BRIEF SUMMARY OF THE INVENTION

Magnetic field producing equipment is used to produce low energy Alternating Current electromagnetic fields. Patients are bathed in these magnetic fields for periods of time ranging from several seconds to several hours. These low energy magnetic fields induce regulatory genes which control the cellular stress response and immune response in all bodily cells, tissues, and organs. This method of regulatory gene induction causes little or no damage to the cell.

The cellular stress response causes a cascade of repair protein production. These proteins reduce damage by scavenging free radicals within the cell. They also repair damage caused by free radicals, solar radiation, and other incidental environmental stresses. The cellular stress response is repeatedly induced, in the subjects' entire body, for the purpose of increasing resistance to infection, and increasing cellular survival of environmental stresses. This treatment is repeated at various intervals throughout the lifetime of the customer. Continuous treatment will lead to longer life span and increased health.

All other known methods of inducing these regulatory genes cause significant cellular damage and are associated with relatively severe side effects. The classic method of stress response induction, thermal stress, requires fourteen orders of magnitude more energy to induce a similar response when compared to magnetic fields.

Since magnetic fields require very little energy to induce these regulatory genes, they cause relatively little or no damage to the cells on the molecular level. This allows for large net gains in cellular damage control performed by the many repair proteins which are produced after exposure. Many currently available products which claim to use magnetic fields for therapeutic purposes, utilize Direct Current magnets. These products do not induce the cellular stress response. These products, as well as those which use AC magnetic fields, are all marketed for use on specific portions of the body and for fixed time periods. None of these products, to my knowledge, claim to induce the cellular stress response. Certainly none have been associated with increased longevity or increased survival in experimental animal.

### BRIEF DESCRIPTION OF THE SEVERAL VIEWS OF THE DRAWING

Not Applicable.

### DETAILED DESCRIPTION OF THE INVENTION

Many types of equipment designed to create low energy magnetic fields may be utilized to induce genes which govern stress and immune response for the purposes of this invention. Two Helmholtz coils oriented on a cylinder at a distance equal to their diameter will produce a relatively equal magnetic field strength at all points within the cylinder between the two coils. The strength of the field is dependent upon the electrical current applied to the coils. The frequency (cycles) of the field will be equal to the AC frequency of the current. This allows for any magnetic field strength, size, and frequency desired.

As stated in “BACKGROUND”, magnetic field and exposure parameters for this application are still being optimized. Optimum results to date have been produced by twenty-minute exposure to eighty milligauss, sixty-hertz, alternating current magnetic fields every twenty-four hours.

Whole body magnetic field exposure is supplied approximately every 24-48 hours. Customers are bathed in these magnetic fields for approximately one half hour per treatment. These low energy magnetic fields induce regulatory genes which govern the cellular stress response and immune system in all bodily cells, tissues, and organs. This method of regulatory gene induction causes little or no damage to the cell.

The cellular stress response causes a cascade of repair protein production. These proteins reduce damage by scavenging free radicals within the cell. They also repair damage caused by free radicals, solar radiation, and other incidental environmental stresses. The cellular stress response is repeatedly induced, in the subjects' entire body, for the purpose of repairing damage on the molecular level. This treatment can be repeated at various intervals throughout the lifetime of the customer. Continuous treatment will lead to longer life span and increased health.

As stated in “BACKGROUND”, this method of regulatory gene induction has not previously been applied to increasing immune response or increasing survival from infectious disease.

1) A method of therapeutically treating patients suffering from reduced immune function, said method comprising: repeated exposure of effected tissues to sinusoidal, alternating current (AC) electromagnetic fields ranging in strength from 2 Gauss to 0.001 Gauss, said fields being produced adjacent to or between conducting media via conduction of appropriate amplitude AC electrical current, said fields being applied to tissues via tissues presence proximate to the conducting media, for a period of time ranging from 1 minute to 24 hours, intervals between treatments ranging from one exposure per minute to one exposure per week.

2) A method of increasing production of beneficial gene products in treated tissues for the purpose of therapeutically treating patients suffering from reduced immune function, said methods comprising: repeated exposure of tissues to sinusoidal, alternating current (AC) electromagnetic fields ranging in strength from 2 Gauss to 0.001 Gauss, said fields being produced adjacent to or between conducting media via conduction of appropriate amplitude AC electrical current, said fields being applied to tissues via tissues presence proximate to the conducting media, for a period of time ranging from 1 minute to 24 hours, intervals between treatments ranging from one exposure per minute to one exposure per week.

3) A method as in claim 2 for maintaining/improving immune system function.

4) A method as in claim 2 for maintaining/improving growth hormone production.

5) A method as in claim 2 for maintaining/improving cellular insulin sensitivity.

6) A method as in claim 2 for use to preventing tumor genesis.

7) A method as in claim 2 for slowing or reversing the effects of aging in the circulatory system.

8) A method as in claim 2 for preventing the on-set, or progression of Alzheimer's Disease.

9) A method as in claim 2 for preventing the on-set, or progression of Parkinson's Disease.

10) A method for inducing the cellular stress response for the purpose of therapeutically treating patients, said methods comprising: repeated exposure of tissues to sinusoidal, alternating current (AC) electromagnetic fields ranging in strength from 2 Gauss to 0.001 Gauss, said fields being produced adjacent to or between conducting media via conduction of appropriate amplitude AC electrical current, said fields being applied to tissues via tissues presence proximate to the conducting media, for a period of time ranging from 1 minute to 24 hours, intervals between treatments ranging from one exposure per minute to one exposure per week.

11) A method as in claim 10 for use to preventing tumor genesis.

12) A method as in claim 10 for maintaining/improving growth hormone production.

13) A method as in claim 10 for maintaining/improving immune system function.

14) A method as in claim 10 for maintaining/improving cellular insulin sensitivity.

15) A method as in claim 10 for slowing or reversing the effects of aging in the circulatory system.

16) A method as in claim 10 for preventing the on-set, or progression of Alzheimer's Disease.

17) A method as in claim 10 for preventing the on-set, or progression of Parkinson's Disease.
